(19) United States
(12) Patent Application Publication
(10) Pub. No.: US 2022/0196624 A1
(43) Pub. Date: Jun. 23, 2022

(54) SYSTEM AND METHOD FOR DETERMINING A GENDER OF AN EMBRYO IN AN EGG

(71) Applicant: LIVEGG (2015) LTD., Ma'anit, R.D. Menashe (IL)

(72) Inventor: Yair Or ADAR, Kvutzat Yavne (IL)

(21) Appl. No.: 17/615,282

(22) PCT Filed: May 30, 2019
(86) PCT No.: PCT/IL2019/050622

§ 371 Date: Nov. 30, 2021

(51) Int. Cl. G01N 33/08; A01K 45/00; G01N 21/3563

(52) U.S. Cl. CPC G01N 33/085; G01N 21/3563; A01K 45/007

(57) ABSTRACT

The present disclosure concerns a system (100) for determining a gender of a fertile egg. The system utilizes measured data (20) indicative of radiation responses of an egg to illumination with at least two wavelengths. The radiation responses include transmissions of the illumination through at least a portion of the egg and/or reflections of the illumination from the egg. The system (100) is configured and operable to process the received measured data (20), to determine the variation over time of a relation between the intensities of the radiation responses at the at least two wavelengths, and to generate gender data based thereon.

### TECHNOLOGICAL FIELD

The present disclosure relates to the field of eggs monitoring, such as, but not limited to, poultry eggs.

### BACKGROUND

In the poultry industry, in particular the chicken industry, discrimination between poultry eggs on the basis of some observable quality is a well-known and long-used practice.

Automatic egg examining devices that utilize the transparency of the egg in order to differentiate between fertilized and unfertilized eggs have been developed over the years. These devices comprise emission means for emitting a light beam in the direction of an egg to be examined, receiving means for receiving the light beam passing through the egg, and means for processing data regarding the light beam received by the receiving means so as to determine the status of the egg.

Some techniques for use in examining eggs and enabling monitoring the incubation process are described in the following patent publications WO15052626, WO15145435, US2017082590, all assigned to the assignee of the present application. According to these techniques, the incubation tray includes a tester unit including plurality of inspection modules in the enclosure associated with the plurality of egg placements. Each inspection module includes radiation emitter(s) and sensor(s), and is configured and operable for inspecting the respective egg by irradiating the egg with radiation and measuring a radiation response of the eggs, giving rise to measured data indicative of condition of the egg. The measured data may be processed to determine dynamic and static parameters of the radiation response from which a physiological development stage, and growth of the embryos within the eggs can be estimated.

Identifying non-fertile or eggs with dead embryos is obvious to have a huge value in the poultry industry, though identifying the gender of the embryo has also significant value in the industry. Some species of chickens are bred for males, for their meat, and other species for females, for their eggs.

Many attempts were made to distinguish between males and females embryos at early stages after the laying of the egg. Such techniques include Fourier transform infrared spectroscopy, Raman spectroscopy, magnetic resonance imaging and more.

For example, Galli, Roberta, et al. “Contactless in ovo sex determination of chicken eggs.”, Current Directions in Biomedical Engineering 3.2 (2017): 131-134 describes contactless in ovo sex determination of the domestic chicken. This technique is based on in ovo Raman and fluorescence spectroscopy of blood of eggs.

### GENERAL DESCRIPTION

The present disclosure concerns a system for determining in non-invasive manner the gender of a fertile egg, in particular poultry eggs. To this end, optical measured data non-invasively collected from the egg is provided and analyzed. The measured data to be analyzed includes radiation response of the egg to illumination of at least one wavelength. Such radiation response includes reflection and/or transmission of said at least one wavelength by the egg. The measured data may be provided off-line from a storage device (generally, from a measured data provider), where such measured data collected during previously performed measurements is maintained, or in an on-line (real time) mode directly from a measurement system (constituting the measured data provider).

The illumination with at least one wavelength is typically selected from the infra-red (IR) spectrum, preferably the near IR spectrum. The egg is illuminated by one or more wavelengths at about the same location so as to obtain repetitive measurements of radiation responses, namely either reflection or transmission or combination of both for each of the respective illumination wavelengths of the egg on the same spot/location of the egg. The inventor has found that male eggs' response to the illumination is different from that of female eggs. Also, such difference might be expressed in the radiation responses collected during successive incubation intervals.

It is to be noted that measurements of radiation responses are not limited to those of a single illumination wavelength and may be carried out with a plurality of different wavelengths, e.g. broadband illumination. In this connection, it should be understood that the radiation response being collected and analyzed is actually a time function (time pattern/behavior) of such factors of the detected radiation as intensity and/or frequency.

Thus, the responses, either of a single wavelength or a plurality of wavelengths and either at a certain time or over certain time interval of incubation, provide different radiation responses and it was surprisingly found that according to certain features that are derived from the measured data of the detected responses, the gender of the fertile egg can be determined. The measured data indicative of/corresponding to the detected responses undergoes model-based analysis based on a fitting procedure using suitable model functions. According to the invention, different models are used for the analysis of different features embedded in the radiation responses. The model function is selected as a functions having a time shape corresponding/matching a time variation of the feature of interest in the measured responses. More specifically, in some embodiments of the invention, the models utilize polynomial functions of different degrees of linearity to obtain the model parameters (polynomial coefficients) corresponding to the features of interest.

The inventors have found that data indicative of one or more of the following features derivable from the radiation responses can be indicative of the embryo's gender: a heart rate relating frequency range (e.g. between 3-5 Hz and more specifically around about 4.5 Hz); an embryo viability frequency component (e.g. between 0.1-0.4 Hz and more specifically around 0.3 Hz); an amplitude/intensity of the detected response.

For example, a polynomial fit of periodical behavior of the radiation response to certain illumination, e.g. a polynomial fit of the time variation of the frequency of the heart rate within the heart rate relating range or the viability signal of the embryo, may provide polynomial coefficients that are used as parameters in a classifying process of gender classifier(s). The gender classifier utility is preprogrammed based on a proper machine learning procedure, as will be described further below.

Preferably, the analysis of each of the above features, or at least the amplitude variation feature, is performed based on the detection of the radiation responses from the egg for at least two different wavelengths. For example, the time evolution of a ratio between the two response signals of the two wavelengths is analyzed.

It should be noted that the terms radiation response and illumination response may be interchangeably used throughout the application.

Accordingly, a first aspect provided by the present disclosure concerns a monitoring system for use in determining a gender of a fertile egg. The system comprises data input utility configured to receive measured data from a measured data provider (measurement system or external storage device). The measured data comprises data indicative of radiation response of an egg to illumination of at least one wavelength. The radiation response includes at least one of transmission of the illumination through at least a portion of the egg and reflection of the illumination from the egg. The system includes a data processor and analyzer utility configured and operable to process the received measured data and determine, in the radiation response, data indicative of one or more of the following features: a heart rate relating frequency range, an embryo viability frequency component, and an amplitude of the radiation response, and perform model based processing of the data indicative of said at least one feature, and generate gender data based thereon. The system also includes an output utility configured and operable to generate association data comprising the gender data in association with the egg being measured, to thereby enabling utilize the association data for sorting the egg according to the gender.

Optionally, the output gender-relating data may be displayed, and/or may be communicated to a control unit of an eggs' sorting station.

As indicated above, in some embodiments of the monitoring system, the radiation response comprises at least one of transmission and reflection of the at least one illumination wavelength from an illumination region of the egg.

In some embodiments, the measured data indicative of the radiation response comprises a plurality of data pieces collected over time during a plurality of measurement sessions. Each data piece may correspond to a single sample of radiation response collected at a certain time of the incubation. The sample, for example, may be a collection of data of radiation response over a time period of several seconds, e.g. 10-120 seconds.

In some embodiments, the data indicative of the at least one of the above features comprises a time variation of the respective feature corresponding to a time signature of said feature. The time signature of the feature, for example, may correspond to a certain function of which its parameters, e.g. coefficients, are indicative of the gender of the embryo.

In some embodiments, the heart rate relating frequency range is 3-5 Hz and the embryo viability frequency component is in a range of 0.1-0.4 Hz.

In some embodiments of the monitoring system, the data indicative of the amplitude of the radiation response comprises an average amplitude of the radiation response detected over time. From each data piece, e.g. a sample of radiation response during the incubation period, an average of the intensity of the radiation response may be determined and utilized as a feature for determining the gender of the embryo.

In some embodiments of the monitoring system, the data processor and analyzer utility includes: (i) a feature extractor utility configured and operable to analyze the measured data and determine said data indicative of the at least one feature; (ii) a classifier utility configured and operable to process the data indicative of the at least one feature by utilizing at least one respective model and predetermined weighting factors and determine likelihood of the gender of the respective egg; (iii) and a gender determination utility configured and operable to determine the gender based on a relation between said likelihood and a predetermined criteria.

In some embodiments of the monitoring system, the classifier utility is configured and operable to process different data pieces of said data corresponding to different segments of the radiation response associated with respective different time intervals of incubation. In other words, the model for determining the gender of the embryo based on the measured data collected over time may use different analysis tools, e.g. different weighting factors, for different data pieces collected in different time intervals over the incubation period.

In some embodiments, the classifier utility comprises a number of classifiers corresponding to a number of the features, each providing the likelihood based on the respective one of the features. The gender determination utility is configured to determine the gender of the egg based on the relation of all the likelihoods and the predetermined criteria being defined by a majority of the likelihoods of the same gender. For example, where the majority of the classifiers determined that its more likely that the embryo is a male, the gender determination utility determines that the embryo of the respective egg is male, and vice versa.

In some embodiments, the model optimization utility configured and operable to define the at least one model to be used in the analysis of each of said at least one feature, e.g. the fitting parameters or the weighting factors of the model.

In some embodiments of the monitoring system, the model optimization utility includes a machine learning processor configured and operable to process data indicative of said at least one feature associated with radiation responses of a training set of eggs of known genders, by performing fitting procedures with the at least one selected model, to thereby optimize model coefficients and their weighting factors.

In some embodiments of the monitoring system, the model is based on a predetermined function, describing a profile of the at least one feature within the radiation response.

In some embodiments of the monitoring system, the model is based on a polynomial function associated with the respective one of the features. For example, the variation of the heart rate frequency or the viability signal may fit to a polynomial behavior, which its coefficients are being utilized in the model for determining the embryo of the egg.

In some embodiments of the monitoring system, the measured data includes at least one of spectral reflection and spectral transmission of the egg. The system further includes a spectral analyzer configured and operable to select the radiation responses for said at least one predetermined wavelength. In other words, the measured data may include a broadband spectral data of a plurality of radiation responses that correspond to a plurality of illuminations of different wavelengths. The spectral analyzer is configured to select one or more desired radiation responses for being used in the model.

In some embodiments of the monitoring system, the measured data includes the radiation responses of the egg to at least two wavelengths.

In some embodiments of the monitoring system, the processor and analyzer utility is configured and operable to determine a relation between said at least one feature in the radiation response of the at least two wavelengths.

In some embodiments, the at least one feature is indicative of the amplitude or the intensity of the radiation response.

In some embodiments, the data indicative of the amplitude or the intensity is an average amplitude or intensity within the radiation response variation over time.

In some embodiments of the monitoring system, the data processor and analyzer utility is configured to determine said relation comprising at least one of the following: a ratio of the measured signals corresponding to the reflection for the two wavelengths; and a ratio of the measured signals corresponding to the transmission for the two wavelengths.

In some embodiments, the measured data received by the input utility includes measured data pieces corresponding to multiple measurements sessions and being collected in different times, for example a series of hourly or daily measurements of the same egg. The control unit selects at least one measurement session for being processed by the processor.

In some embodiments, the monitoring system includes a measurement system operable as the measured data provider. The measurement system includes:

(a) an illuminator comprising at least one light source for illuminating the egg with the at least one wavelength;

(b) a detector unit comprising at least one detector for detecting the radiation response of the egg comprising at least one of egg's reflection and transmission to said at least one wavelength of the illuminations, and generating the measured data indicative thereof.

In some embodiments, the monitoring system further includes an automatic optical inspection (AOI) system for inspecting a plurality of fertile eggs, each contained in an egg cell in an incubation tray. The AOI system includes:

(1) a measurement system configured and operable to perform optical measurements on each egg in the incubation tray and provide, for each egg, measured data indicative of radiation response of the egg to at least one illumination wavelength;

(2) a monitoring system configured and operable according to any one of the above described embodiments; and

(3) a sorting utility configured to utilize the association data to control the sorting of the egg.

Another aspect of the present disclosure provides a sorting station for controlling sorting of fertile eggs progressing on a production line according to their genders, the sorting station comprising: a controller configured to be in data communication with the monitoring system according to any one of the above described embodiments, and an identifier utility configured and operable to identify the egg and the association data of the identified egg, and generate sorting data defining further progress of the egg towards one of two routes. For example, the sorting data may define whether the egg proceeds in the incubation process or whether it is removed from incubation.

### BRIEF DESCRIPTION OF THE DRAWINGS

In order to better understand the subject matter that is disclosed herein and to exemplify how it may be carried out in practice, embodiments will now be described, by way of non-limiting examples only, with reference to the accompanying drawings, in which:

FIGS. 1A-1B are block diagrams of a control system of the invention for use in a monitoring system for determining a gender of an embryo in an egg;

FIG. 2A exemplifies two optical responses collected over time from an egg illuminated by two different wavelengths. The representation of the graph emphasizes the harmonic frequency of about 0.3 Hz. The response is represented by voltages applied by the light detected in the detector.

FIG. 2B shows an illumination response over time of an egg. The representation of the graph emphasize the harmonic frequency of about 4-5 Hz. The response is represented by voltages applied by the light detected in the detector.

FIG. 3 exemplifies a flow diagram of a process for determining a gender of an embryo in an egg according to the present disclosure.

FIGS. 4A and 4B show block diagrams of two examples of the monitoring system of the present disclosure.

FIGS. 5A-5B are examples of a pair of illumination responses of two wavelengths.

FIGS. 6A-6C are non-limiting examples of the behavior of the relation between intensities of the measured illumination responses of eggs of males and females, based on simulations and experiments performed by the inventor.

FIGS. 7A-7D are corresponding pairs of bars diagram and receiver operating characteristics (ROC) graph showing the probability of different parameters of features, derived from the illumination responses, to predict the gender of the embryo.

FIG. 8 is a non-limiting example of a flow diagram exemplifying the process of determining the gender of the embryo of a poultry egg.

FIG. 9A-9B exemplify different degree of linearity fit of variation of features over time of incubation.

### DETAILED DESCRIPTION OF EMBODIMENTS

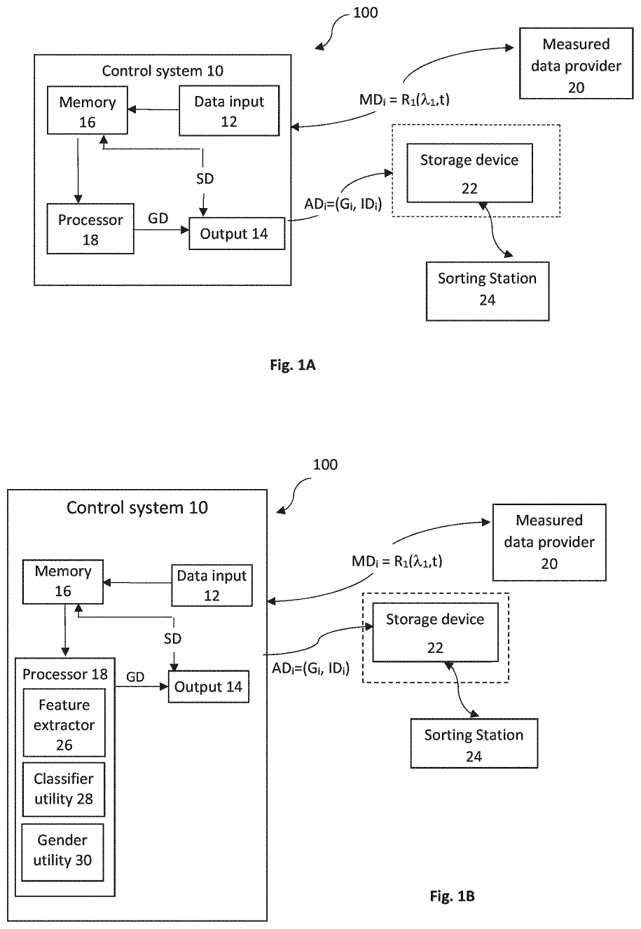

Referring to FIG. 1A, there is illustrated, by way of a block diagram, a monitoring system 100 of the present invention, for use in determining a gender of a fertile egg. The system 100 comprises a control system 10 comprising such main parts/modules (software and/or hardware utilities) as data input and output utilities 12 and 14, memory 16, and a data processor and analyzer utility 18. The monitoring system 100 is configured and operable to be in data communication (e.g. via wires or wireless communication using any suitable technique and communication protocols) with a measured data provider 20. The measured data provider 20 may be a storage device to which the monitoring system 100 has access (e.g. via a communication network), and which stores measured data previously collected (so-called “off-line” mode of operation of the monitoring system) by performing optical measurements on fertile eggs, e.g. during incubation period. Alternatively or additionally, the storage device 20 may be associated with (part of) a measurement system. The measured data may be directly provided to the monitoring system during the measurements performed on the eggs (on-line mode).

For the purposes of the present invention, the measured data MDᵢ for each i-th egg includes at least one optical responses R₁ of the egg to illumination of at least two one wavelength λ₁, each radiation response being collected over time. The optical response(s) for the at least one wavelength include(s) the egg's reflections to the wavelength and/or the egg's transmission of the wavelength of illumination. This will be described more specifically further below. Generally, however, the raw measured data provided to the control system 10 may include a spectral response (reflection and/or transmission) of the egg to a plurality of wavelengths (e.g. broadband illumination), and the control system 10 may select data related to at least one wavelength for further processing according to the technique of the invention, as will be described more specifically further below.

Thus, the data input utility 12 receives and stores the measured data MDᵢ for each i-th egg being monitored. The data processor and analyzer utility 18 is configured and operable (preprogrammed) to process the measured data, and determine in/extract from the radiation response, data indicative of one or more of the following features: a heart rate relating frequency range, an embryo viability frequency component, and an amplitude of the radiation response, and perform model based processing of the data indicative of said at least one feature, and perform model based processing of the data indicative of the feature(s), and generate gender data GD based thereon. The data indicative of the features may include parameters corresponding to the responses for the two wavelengths (e.g. coefficients of polynomial fits of certain features' profiles over time of the illumination responses, a relation between the transmissions of two wavelengths and/or a relation between the reflections of two wavelengths). These characteristics of the data of each feature or the relation between two or more features is indicative of the gender G of the egg, i.e. the data indicative of the feature(s) is different for the two genders. The output utility 14 generates respective association data ADᵢ corresponding to the gender G of the i-th egg. The association data AD may be stored in the memory 16.

Sorting data can be extracted from the memory by the output utility 14 at any later time, and/or the sorting data may directly, or later upon demand/request, communicated to a storage device 22. The storage device 22 may be part of or be in data communication with a sorting station 24 sorting eggs according to their gender. As also noted in the figure, the association data also includes identification data of the respective egg. This may be a direct mark/code on the egg and/or on the respective cell of the incubation tray where the egg is located, and may be imaged/read during the measurement itself, and/or during the sorting procedure itself; and/or may include a location data (e.g. row and column data) of the respective cell in the incubation tray where the egg is located, as the case may be. Generally, the sorting data used in the sorting procedure utilizes the determined gender data in association with the egg's ID.

FIG. 1B differs from FIG. 1A by showing a non-limited example of a more elaborated embodiment of the data processor and analyzer utility 18, including its different modules. In this embodiment, the processor 18 includes a feature extractor 26 that is configured to extract one or more of the above features from the measured data (radiation responses) MDᵢ. For example, the feature extractor is configured to extract the frequencies of the heart rate of the embryo from each sample of the measure data and use the collection of extracted features for identifying and determining parameters of the variation of the heart rate over time to be used in a classifier utility 28. The classifier utility 28 is configured and operable to process the data indicative of the at least one of the above features (e.g. time variation of the feature) by utilizing at least one respective model and predetermined weighting factors and determine likelihood of the gender of the respective egg. Thus, the Classifier 28 receives the data indicative of the feature(s) and the respective model(s) and determines likelihood of each egg for being a male or a female based on the respective feature. A gender determination utility 30 is configured to receive the likelihood of each of the plurality of the above features from the classifier utility 28 and determine the gender of the embryo in the respective egg based thereon.

Thus, the inventor has found that response of illumination of a fertile egg of poultry has an observable signature, and by the right analysis, this signature can assist in distinguishing, non-invasively, between a male's egg and female's. When the egg is illuminated, for example with illumination having a wavelength in the range of 600-1400 nm, a response of the illuminated region on the egg, namely the reflection and/or the transmission of the illumination, exhibits at least two periodical behaviors, unless the embryo is not alive.

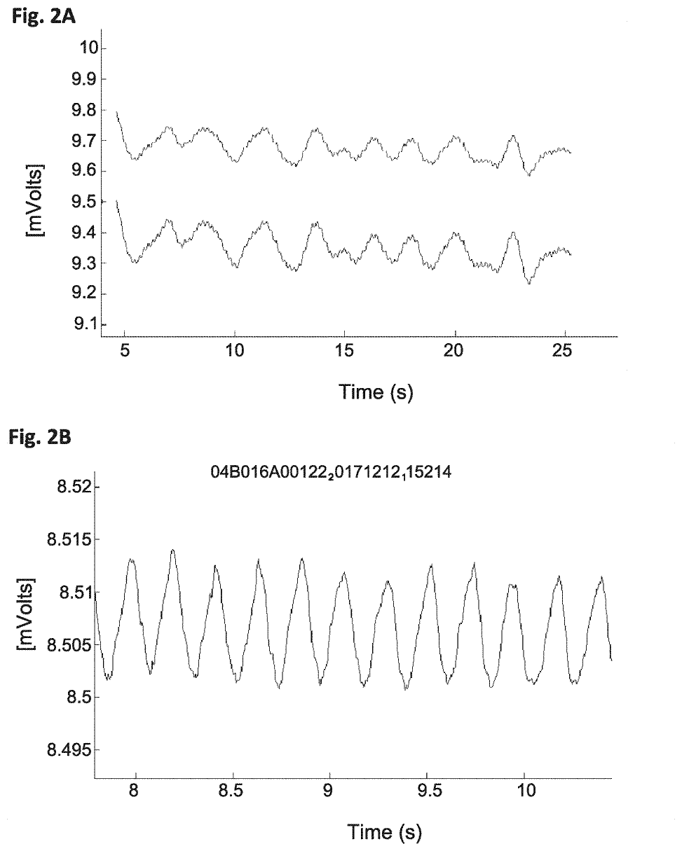

The present invention takes advantages of the earlier techniques of the inventor, described in the above indicated patent publications, e.g. WO15052626, according to which the periodical behaviors are typically exhibited with frequencies 0.1-0.4 Hz at the initial incubation period (e.g. at the fifth-seventh day of the incubation period) and are indicative of vital signs of an embryo in the egg, and with the frequencies of about 3-5 Hz indicative of a heart rate identifiable at day 3 observing increase of amplitude over time during the incubation period. These periodical responses in the frequencies of about 0.3 Hz and of about 4-5 Hz are shown, respectively, in FIGS. 2A and 2B. In these examples, the provided responses are transmissions of illuminations with wavelengths of 850 nm and 740 nm in the upper and lower graphs in FIG. 2A respectively, and 850 nm in the graph of FIG. 2B.

The inventor has now also found that one of the features that distinguishes between a male and a female of an embryo in an egg is a relation, e.g. ratio, between the optical responses of the same type (reflection or transmission) of the illuminated region/spot on the egg to illumination of two different wavelengths.

It should be noted, that preferably, for the gender determination, the optical response is to be considered as a time function of the optical signal from the egg. More specifically, this may be at least one continuous measurement session, or a plurality of time separated measurement sessions, e.g. one or measurements are successively performed with a few seconds duration, during the incubation period. Also, the optical response data used for gender determination may include data indicative of a time changes/dynamics in the relation between two responses for different wavelengths, as well as the same for transmission and reflection modes.

The inventor has shown that the technique of the invention provides for determining the embryo gender already from the 8-th day of incubation. It should be understood that the stronger the detected (measured) signals the higher is the precision of embryo gender determination. In the experiments conducted by the inventor, the sufficiently strong signals were obtained on the 10-th day.

Thus, provided in the present disclosure is a system for monitoring a fertile egg for determining the gender of the embryo in the egg. Further to such determination, the eggs can be sorted by a sorting unit, also provided by this disclosure, to eggs containing male embryos and eggs containing female embryos.

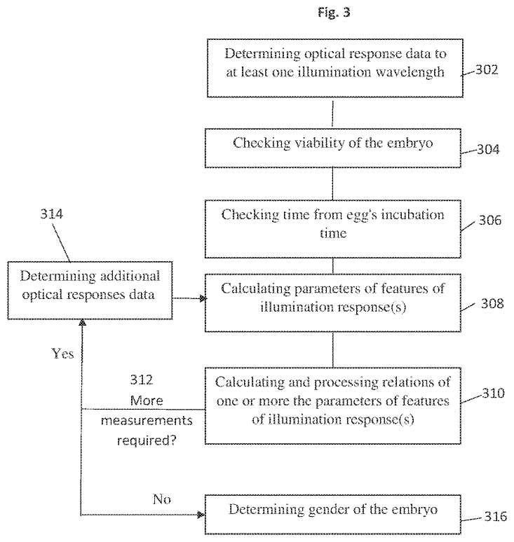

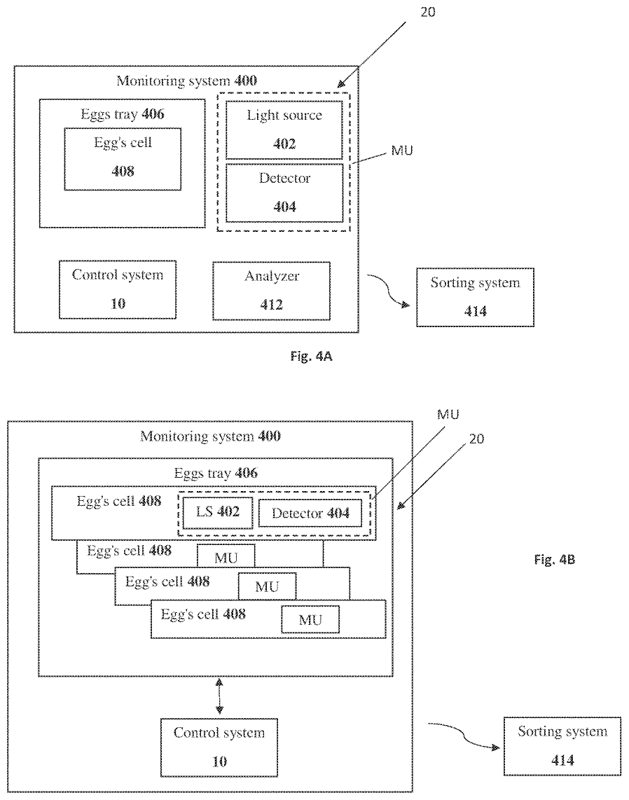

FIG. 3 exemplifies a flow diagram of a method to determine a gender of an embryo in an egg according to the present disclosure. It is to be understood that the sequence of the steps is not limiting and some of the steps are interchangeable. Furthermore, it is noted that not all the steps are mandatories, since the method can be carried out omitting some of the steps.

As described in FIG. 3, at first, data of an optical response of an egg to illumination of a number i of wavelengths λ (i≥1), R_{1 . . . i}(λ_{1 . . . i}, t), is provided—step 302. As described above, this data can be received from a measured data provider (storage device that stores a previously collected measured data, or it can be received directly from a measurement unit, taking the measurements in real-time).

The measured data indicative of at least one detected radiation response (response signal) can be used to determine whether the embryo is alive or not—step 304. To this end, the technique described in the above-indicated patent publications can be used, namely identifying harmonic behaviors of the optical response, typically having the frequencies in a range of 0.1-0.4 Hz, e.g. about 0.3 Hz, the existence of which indicates the viability of the embryo. In this case, the detection of this condition may be used to activate/initiate further measurements/or may be used in combination with the other measured data for at least one other wavelength providing together measurements for one or more wavelengths with regard to living embryos, to perform the above-described procedure to determine their gender. As noted above, each egg has its unique identification (e.g. via ID of the respective cell) included in/provided with the measured data, which among other details, may include data indicative of the time passed since the egg was laid or the respective incubation time—step 306. The time parameter might be considered for the process of the determination of the gender of the embryo since the optical response varies in time, such that the profile of the expected response, for both, males and females are differentiate between days.

Thus, data indicative of various features (e.g. periodical behavior over time, relation of intensities over time) in the detected optical responses (measured signals) for all of the determined illuminating wavelengths is determined/calculated (step 308). For example, one pair of optical responses, R₁ and R₂, of the same type (transmission or reflection) for at least one pair of wavelengths, is processed to determine one or more parameters according to the relation between the responses or according to a function corresponding to a variation profile of each of the responses f(R₁, R₂). The relation of the one or more parameters that are derived from features of the optical responses are processed and calculated—step 310. The measured signal includes detected light intensity of the optical response to illumination. A feature based on a relation between two light intensities for two different wavelengths may actually be based on any suitable function, e.g. a difference between the two intensities. Preferably, such relation may be a ratio between two measured optical responses R₁/R₂. The data selected for analysis may be based on the harmonic responses of the mostly informative frequencies, e.g. the harmony of about 0.1-0.4 Hz or about 3-5 Hz. To improve the accuracy of the measurements, a series of optical responses collected over time is considered which determine together the value of the feature of interest, using a statistical manipulation, e.g. an average value of all the collected features from the optical responses.

In case more data is required for the determination of the gender of the embryo, namely more pairs of measurements are required—step 312, additional optical responses data may be determined 314 Rₖ(λ_{k . . . 1}, t) in a generally similar manner to the data received in step 302 (e.g. selected from the generally broadband spectral response). It should be noted that the measured signals for different optical responses used for determining the data indicative of the features of interest preferably correspond to the illumination/response of the same portion/region/spot of the egg and under the same or substantially similar measurement conditions (measurement scheme (light propagation scheme, polarization effects, etc.), environmental conditions, etc.). This is because different portions of the egg might respond differently to the same illumination (wavelength, conditions), e.g. due to different scattering properties of the shell/embryo.

It should be noted that data indicative of the optical response may include measured harmonics or intensity (amplitude of detected light signal), or generally any other parameter/characteristic being in a known relation with the intensity, i.e. any functional/parameter associated with the measured intensity. This may be a change of intensity, rate of change, energy. In the description below, such data indicative of the optical response is at times referred to as intensity.

The new data obtained in step 314 is processed in a similar manner according to steps 308 and 310 as described above.

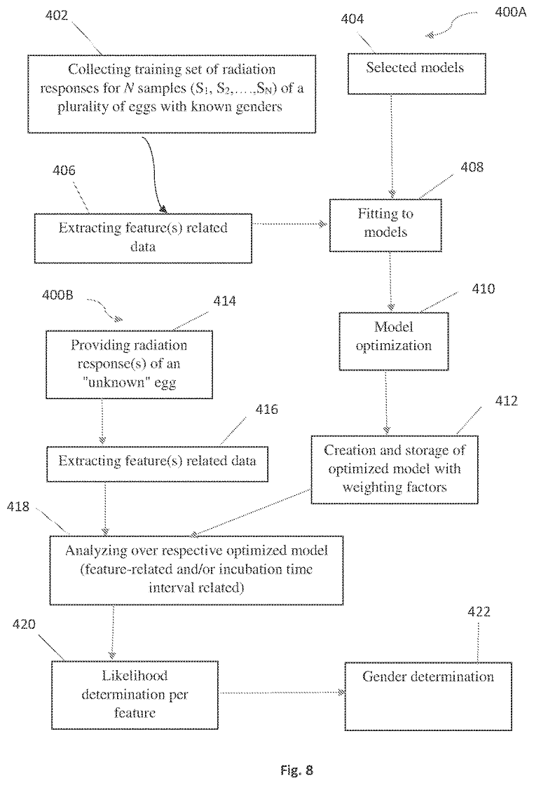

FIG. 8 is a non-limiting example of a method of the invention for determining the gender of the embryo of a poultry egg. FIG. 8 actually illustrates two flow diagrams, a flow diagram 400A for creating model data to be used in the determination of the gender of a so-called “unknown egg”, and a flow diagram 400B for determining the gender of the unknown egg utilizing this model data.

The model data creation process 400A includes collection of measured samples of radiation responses of a plurality of eggs of the known genders, e.g. each egg being sampled N times for providing N samples during the incubation period (step 402). One or more selected predetermined models are provided and stored in a memory utility (step 404).

As indicated above, the selected models are based on functions having time shapes corresponding/matching time variations of the respective feature of interest in the measured responses, e.g. heart rate relating frequency range and/or embryo viability frequency component and/or amplitude of the radiation response. Preferably, the models utilize polynomial functions of different degrees of linearity.

The samples of the radiation responses are analyzed in a feature extractor utility (step 406) which is configured and operable to extract from the radiation responses the above features related data, e.g. deriving a frequency from each sample of the heart rate or the viability signal of the embryo, or extracting an average amplitude/intensity from each sample. The data indicative of the extracted features are fitted to the respective models in an iterative procedure (step 408), and the model parameters corresponding to the features of interest are optimized by the weighting factors of the model coefficients (step 410). Such model parameters may include coefficients of polynomial fits of variation of features over time, relation of function coefficients between one or more features, relation of function coefficients between features derived from samples of radiation responses of different wavelengths or any combination thereof. The model is then fed by these parameters and is being optimized, e.g. weighting factors of relations between the parameters of a machine learning algorithm (such as a random forest algorithm). The optimized model is stored and ready for use in a classification process of samples of eggs with unknown genders (step 412).

Each unknown egg, i.e. an egg whose gender is unknown and is to be determined during the incubation period, is monitored by the above described monitoring system. The main operational steps of this process are exemplified by the flow diagram 400B. The egg is measured/sampled to obtain one or more samples of the radiation responses in any of the above described measurement schemes (reflection and/or transmission, one or more illumination wavelengths, multiple time spaced measurement sessions, etc.). Thus, the measured data of the egg being monitored is provided (step 414), e.g. accessed in the memory. This measured data undergoes the feature extraction process (step 416) to determine the features' related data in the same manner as explained above with respect to the training set samples. The extracted feature related data is processed/analyzed (in the respective, feature-related classifier utility) using the respective optimized model in a fitting procedure (step 418) to determine a likelihood for the gender (step 420). In other words, according to each feature, the model provides a likelihood of the respective egg of being a male or a female. Based on a combination of likelihoods determined from each feature, the gender of the embryo in the egg is determined (step 422) by a gender determination utility based on a relation between the likelihood and a predetermined criteria. The combination of likelihoods may be according to a predetermined weighting factors, giving each feature based likelihood a different weight in the final decision of the gender.

Thus, based on the parameters of the features derived from the measured signals (e.g. coefficients of polynomial fits of certain features over time or ratio between measured signals), the gender of the embryo can be determined, though, as described above, more measurements can be obtained, by using different wavelengths, and/or taking more measurements from different portions of the egg, and/or performing both reflection and transmission mode measurements e.g. for increasing the accuracy of the gender determination, and analyzing measured data corresponding to different successive intervals of the incubation period. In some embodiments, further verification can be performed by analyzing a relation (ratio or difference) between responses for transmission and reflection mode measurements. More specifically, the difference/ratio between (R₁/R₂)_{ref1} and (R₁/R₂)ₜᵣₐₙₛ (taken concurrently or almost concurrently, or successively) for the same pair of wavelengths and with the same illumination spot is different for different genders.

FIGS. 4A and 4B show block diagrams of two examples of the monitoring system of the present disclosure. In these examples, the monitoring system 400 includes a control system 10 and a measurement system 20 (constituting measured data provider 20 in FIG. 1) which are integral in a common system associated with/including an eggs' tray 406. The eggs' tray 406 includes a plurality of egg cells 408, each egg cell being configured to contain one egg that is identifiable either by its location on a specific egg cell and/or according to an identification mark imprinted on the egg's shell/cell.

In the example of FIG. 4A, the measurement system 20 includes a measurement unit MU, including a common light source unit 402 and a common detection unit 404, which is used to perform measurements on each of the eggs. The measurement system is configured and operable in reflection and/or transmission modes, and with one or more different wavelengths (e.g. broadband light). In the example of FIG. 4B, the measurement system 20 includes a plurality of measurement units MU (each operable in reflection and/or transmission modes), each measurement unit being integral with the respective one of the egg cells, and including a light source unit 402 having at least one light source configured for illuminating an egg with at least one wavelength, and at least one detector 408.

In both examples, the measured data from the detection unit(s) is communicated to a control system 10 and may be stored in a memory.

It should be understood that the light source unit 402 is capable of generating one or more illumination beams of one or more different wavelengths and the egg's responses (transmission and/or reflection) to such illuminations are detected by the detection unit(s). The measurements (illumination and detection) may be performed almost concurrently (e.g. in intervals of milliseconds, microseconds, nanoseconds or picoseconds). A measurement can last a few seconds, e.g. 1, 2, 3, 4, 5, 6, 7, 8, 9, 10, 15, 20, 25 or at times 30 seconds and more. An exemplary measurement can, for example, be carried out by alternatingly illuminating a specific portion of the egg with one or more wavelengths (e.g. by modulating the light source operation), each interval of illumination may last a few microseconds, though the total of the measurement, namely all intervals together, may last for 10 or 15 seconds.

The collected measured data is then processed and analyzed by the control system 10, as described above, to determine, for each illumination response of a wavelength, one or more parameters of features of the optical responses for at least one transmission and reflection modes. Based on the calculated parameters, the control system 10 (its processor utility) determines the gender of the embryo, and the result is transmitted to a sorting unit controller 414 enabling association of the egg-relating result with the egg's ID. As described above, the egg's ID may be a mark on the egg and/or on the egg cell, and/or may be location data of the respective cell or of the respective measurement unit. For example, the measured data piece may be associated with the egg via an ID of the respective detection unit.

The output data indicative of the result may also be displayed. For example, the data conveyed to the sorting controller may include egg relating gender and ID, enabling sorting the eggs to males and females.

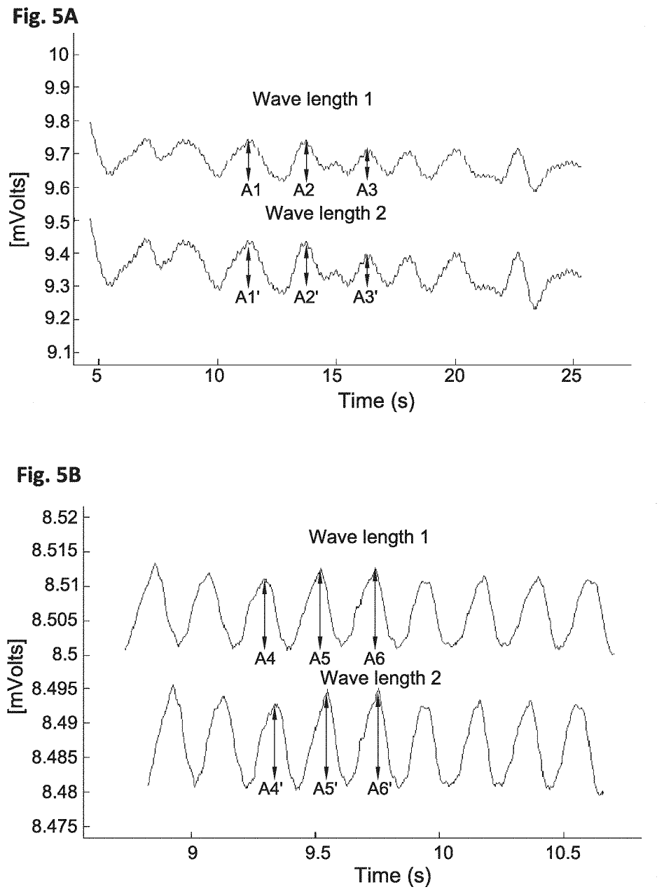

FIGS. 5A-5B are examples of a pair of optical responses of two wavelengths collected over about 25 seconds and about 10 seconds respectively. In FIG. 5A the observable harmonic frequency is about 0.3 Hz, and the intensity (generally measured signal) may be determined according to at least one of the amplitudes A1, A2 and A3 or according to any combination/function thereof for wavelength 1, and A1′, A2′ and A3′ or according to any combination/function thereof for wavelength 2. In a similar manner, the observable harmonic frequency in FIG. 5B is about 4-5 Hz and the intensity may be determined according to at least one of the amplitudes A4, A5 and A6 or according to any combination function thereof for wavelength 1, and A4′, A5′ and A6′ or according to any combination function thereof for wavelength 2.

In some embodiments, the feature indicative of the gender of the embryo may be an average of the intensity over the period of time of the sample. For example, in FIG. 5A, the average intensity of wavelength 1 is about 9.7 mVolts and the average of wavelength 2 is about 9.4 Mvolts, and in FIG. 5B the average intensity of wavelength 1 is about 8.505 mVolts and the average of wavelength 2 is about 8.487 Mvolts. Any relation between the average of intensities of different figures may provide a feature that is indicative of the embryo gender to be used in the classification model/process.

Data from two incubation cycles was collected. The first cycle included 81 eggs incubated in 13 different trays from which 25,379 samples of radiation responses were taken; the second cycle included 213 eggs incubated in 40 different trays from which 53,932 samples were taken. From the total sum of the eggs, 151 males and 143 females hatched. The data of this experiment was taken in the time interval of day 5 to day 18 of incubation. The following coefficients were extracted from the samples of this experiments:
5 polynomial fit coefficients from frequency estimation module of radiation response of a first wavelength (˜0.3 Hz);
5 polynomial fit coefficients from frequency estimation module of radiation response of a second wavelength (˜0.3 Hz);
5 polynomial fit coefficients from frequency estimation module of radiation response of a first wavelength (˜4.5 Hz);
5 polynomial fit coefficients from frequency estimation module of radiation response of a second wavelength (˜4.5 Hz);
2 coefficients from line fit for amplitude of a radiation response of a first wavelength;
2 coefficients from line fit for amplitude a radiation response of a second wavelength;
2 coefficients—ratio amplitude coefficients of the radiation responses of the first and second wavelengths;
4 additional Features:
   Standard deviation (Std) of the amplitude of a radiation response of a first wavelength at days 7-12, and Std of the amplitude at days 13-16.
   Std of the amplitude of a radiation response of a second wavelength at days 7-12, and Std of the amplitude at days 13-16.

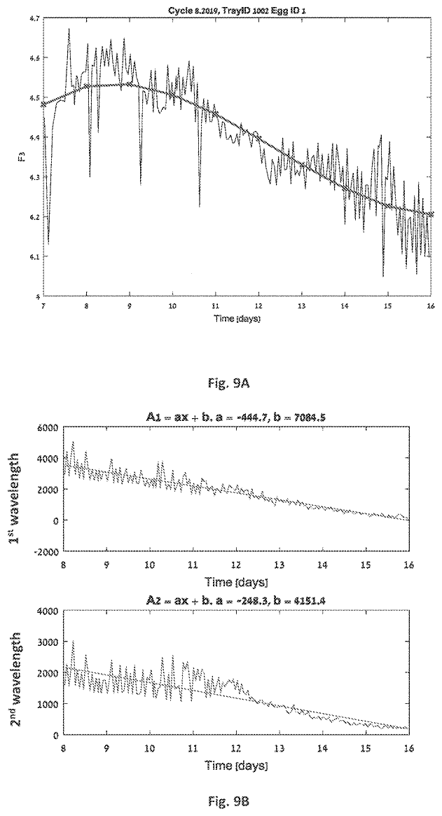

FIG. 9A-9B exemplify different degree of linearity fit of variation of features over time of incubation. FIG. 9A shows a non-limiting example of a 3^{rd} degree polynomial fit of the heart rate frequency variation over time of incubation, from which 5 parameters are extracted: 4 polynomic coefficients and a median value of all the sampled frequency during incubation period. The frequency of the heart rate is extracted from each sample of the respective egg during incubation (e.g. by Fourier transform tools) to receive the collection of heart rate frequencies that are used for the fitting. FIG. 9B exemplifies two linear fits of the amplitudes of radiation response, of two different wavelengths, collected during the incubation period. The linear fit provides two parameters (2 coefficients), and additional 4 parameters are determined by calculating the variance of the amplitudes between day 8-12 and between day 12-16, and by calculating the ratio of the corresponding coefficients between the fit of the first wavelength and the second wavelength.

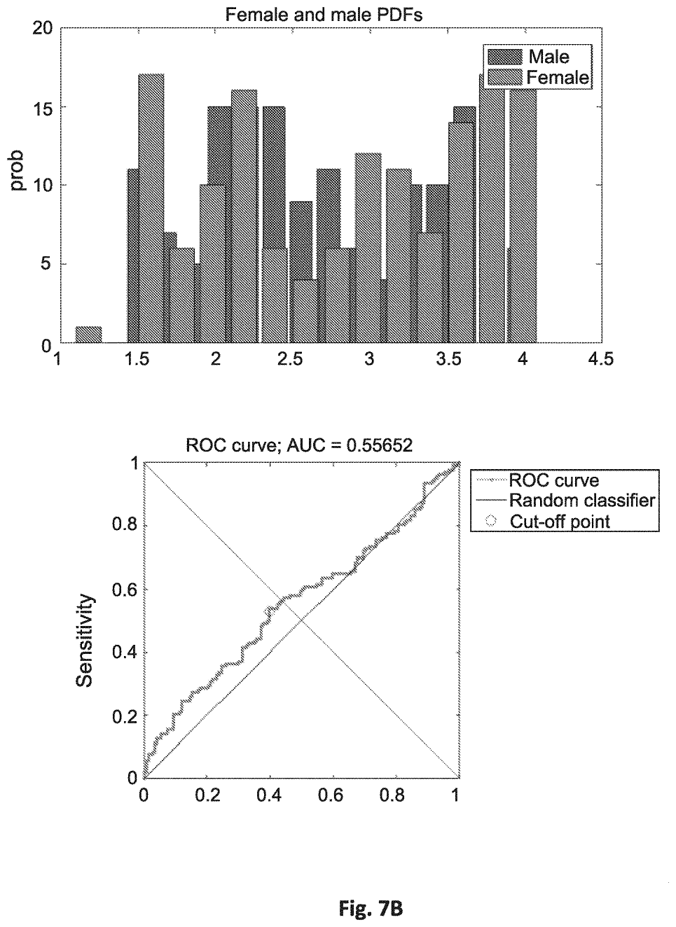

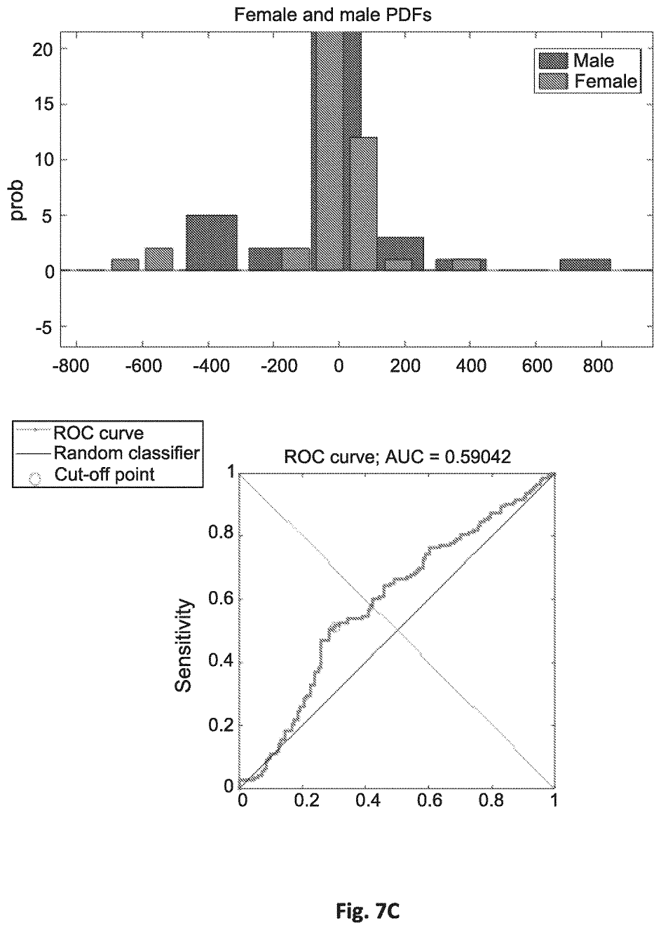

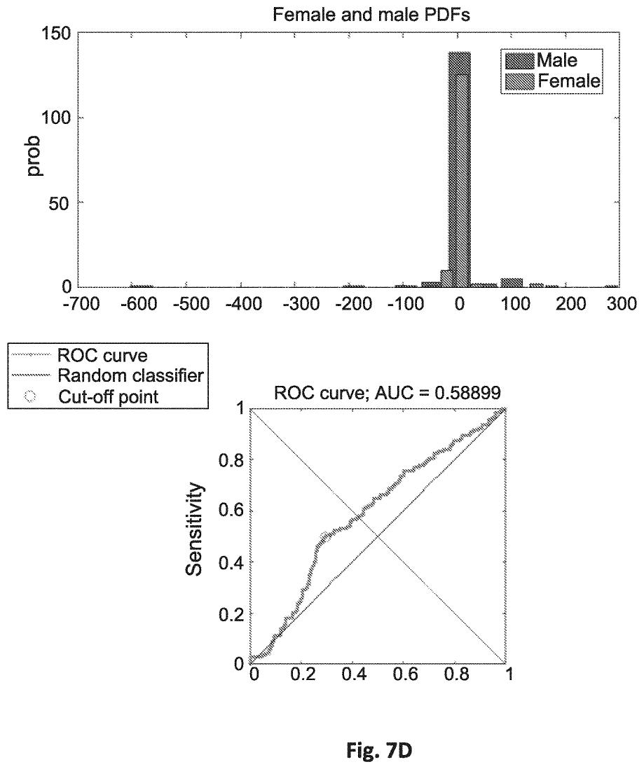

FIGS. 7A-7D are corresponding pairs of non-limiting examples of bars diagram and receiver operating characteristics (ROC) graph showing the probability of different parameters of features, derived from the radiation responses, to predict whether the embryo is a male or a female. The data presented in FIGS. 7A-7D derived from the experimental data described above. FIG. 7A exemplifies the probabilities of different values of the one of the coefficients of a 3^{rd} degree polynomial fit of the variation profile of the viability harmony of the embryo over time (i.e. the variation of the frequency about 0.3 Hz over time of the incubation). Each color of the bars in the graph corresponds to different gender. Similarly to FIG. 7A, FIG. 7B exemplifies the results for additional coefficient of the polynomial fit of the viability harmony. FIG. 7C exemplifies the results a coefficient of a 3^{rd} degree polynomial fit of the variation profile of the heart beat harmony of the embryo over time (i.e. the variation of the frequency about 4 Hz over time of the incubation) and FIG. 7D exemplifies the results for additional coefficient of the polynomial fit of the heart beat harmony, wherein FIGS. 7C-7D exemplify the modeling of data samples collected between days 10-13 of incubation.

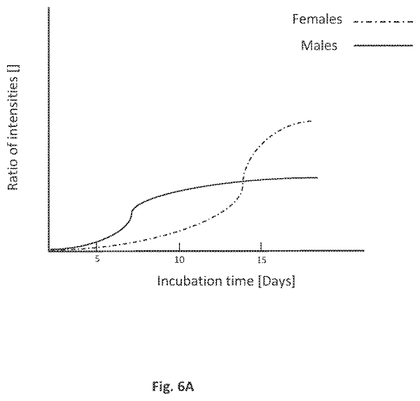

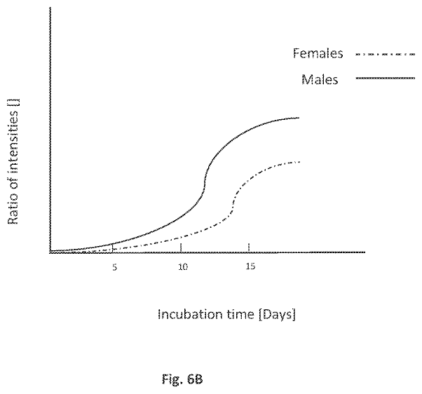

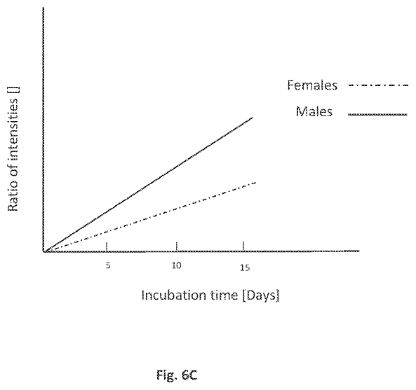

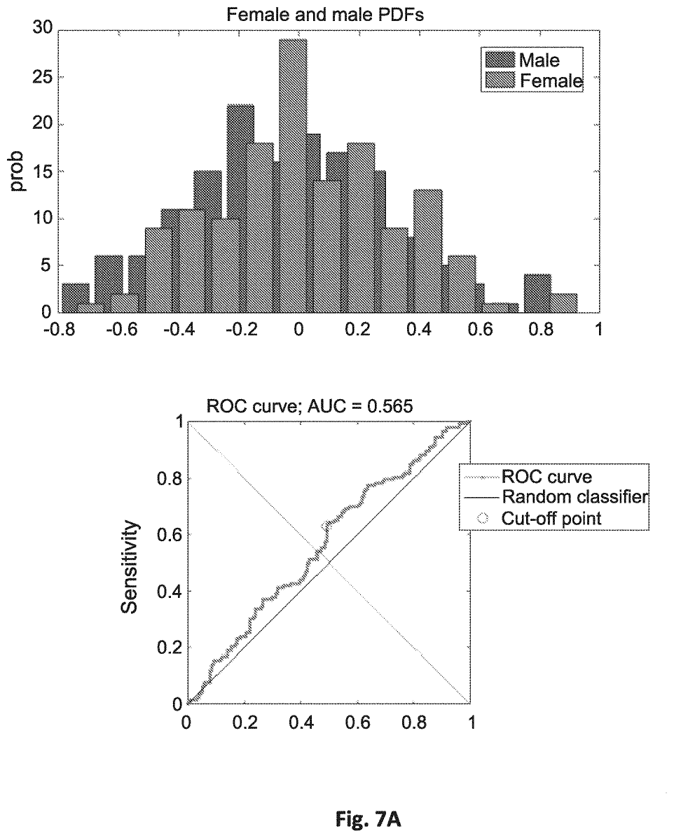

FIGS. 6A-6C are non-limiting examples of expected time variations of the ratio between intensities of the detected optical responses over time between males and females of different poultry species. FIG. 6A shows an example in which the ratio of intensities in males eggs is higher than that of the females up to a critical point (approximately between day 13 and 14 from incubation), after which the ratio of intensities of the females becomes higher. Therefore, eggs whose measured ratio of intensities follows the dashed line, will be sorted as females, and those following the plain line will be sorted as males. It should be mentioned that for other species the behavior of measurements of ratio of intensities can be the opposite to that presented in FIG. 6A, namely at the first days from incubation (i.e. initial incubation period), the relation (ratio) between the optical responses of the same type for different wavelengths of the females is higher and inversed during incubation time. FIGS. 6B and 6C show a polynomial and linear behavior of the relation (ratio) of measured signals, respectively. Each of the figures exemplifies a behavior of the relation between the measured signals such that from the very first days of incubation (i.e. initial incubation period of 2 or 3 days), the ratio for the males is higher than that of the females. Thus, eggs corresponding to ratio between the measured signals that follows the dashed line, will be sorted as females, and those following the plain line will be sorted as males.

As can be seen from these examples, the male and female embryos differently respond to the illumination of the same pair of wavelengths (i.e. characterized by different relation between the two responses of the pair) actually from a very initial time of the incubation period, and this difference can be clearly identified on for example 8-th-10-th day of incubation. As described above, this depends mainly on the ability of the measurement technique to obtain measured signals of the desired strength to determine the relation between the two measurements of different wavelengths.

Thus, the present invention provides a novel approach for determining in a non-invasive manner the gender of a fertile egg. The technique of the present invention can be performed in using relatively simple measurement schemes.

1. A monitoring system for use in determining a gender of a fertile egg, the monitoring system comprising a control system comprising:
data input utility configured for data communication with a measured data provider to receive therefrom measured data indicative of a radiation response of the egg to illumination of at least two different wavelengths;
a data processor and analyzer utility configured and operable to analyze the measured data to determine a relation between amplitudes of the radiation responses of the at least two wavelengths, and a gender determination utility configured and operable to generate gender data based on said relation; and
an output utility configured and operable to generate association data comprising the gender data in association with the egg being measured, to thereby enable to utilize the association data for sorting the egg according to the gender.

2. The monitoring system of claim 1, wherein the radiation responses comprises at least one of transmissions and reflection of said at least two illumination wavelength from an illuminating region of the egg, said data processor and analyzer utility being configured to determine said relation comprising at least one of the following: a ratio of the measured signals corresponding to the reflections for the two wavelengths; and a ratio of the measured signals corresponding to the transmissions for the two wavelengths.

3. The monitoring system of claim 1, wherein the measured data indicative of the radiation response comprises a plurality of data pieces collected over time during a plurality of measurement sessions.

4. The monitoring system of claim 1, wherein said data processor and analyzer utility is further configured and operable to analyze the measured data to determine data indicative of at least one of the following features: a heart rate relating frequency range, and an embryo viability frequency component, the measured data comprises a time variation of the respective at least one feature corresponding to a time signature of said at least one feature.

5. The monitoring system of claim 4, wherein said heart rate relating frequency range is 3-5 Hz.

6. The monitoring system of claim 4, wherein said embryo viability frequency component is in a range of 0.1-0.4 Hz.

7. The monitoring system of claim 1 wherein said data indicative of the amplitude of the radiation response comprises an average amplitude of the radiation response detected over time.

8. The monitoring system according to claim 4, wherein the data processor and analyzer utility comprises:
a feature extractor utility configured and operable to analyze the measured data and determine said data indicative of said at least one feature; and
a classifier utility configured and operable to process said data indicative of the at least one feature by utilizing at least one respective model and predetermined weighting factors and determine likelihood of the gender of the respective egg;
said gender determination utility being configured and operable to optimize the gender data based on a relation between said likelihood and a predetermined criteria.

9. The monitoring system according to claim 8, wherein said classifier utility is configured and operable to process different data pieces of said data corresponding to different segments of the radiation response associated with respective different time intervals of incubation.

10. The monitoring system according to claim 8, wherein said classifier utility comprises a number of classifiers corresponding to a number of the features, each providing the likelihood based on the respective one of the features; and said gender determination utility is configured to determine the gender of the egg based on the relation of all the likelihoods and the predetermined criteria being defined by a majority of the likelihoods of the same gender.

11. The monitoring system according to claim 8, further comprising a model optimization utility configured and operable to define the at least one model to be used in the analysis of each of said at least one feature.

12. The monitoring system according to claim 11, wherein said model optimization utility comprises a machine learning processor configured and operable to process data indicative of said at least one feature associated with radiation responses of a training set of eggs of known genders, by performing fitting procedures with the at least one selected model, to thereby optimize model coefficients and their weighting factors.

13. The monitoring system according to claim 12, wherein the model is based on a predetermined function, describing a profile of said at least one feature within the radiation response.

14. The monitoring system according to claim 13, wherein the model is based on a polynomial function associated with the respective one of the features.

15. The monitoring system of claim 1, wherein the measured data comprises at least one of spectral reflection and spectral transmission of the egg, the system further comprising a spectral analyzer configured and operable to select the radiation responses for said at least one predetermined wavelength.

16-20. (canceled)

21. The monitoring system according to claim 1, comprising a measurement system operable as the measured data provider, the measurement system comprising:
an illuminator comprising at least one light source for illuminating the egg with the at least two wavelengths;
a detector unit comprising at least one detector for detecting the radiation response of the egg comprising at least one of egg's reflection and transmission to said at two wavelengths of the illuminations, and generating the measured data indicative thereof.

22. An automatic optical inspection (AOI) system for inspecting a plurality of fertile eggs, each contained in an egg cell in an incubation tray, the system comprising:
a measurement system configured and operable to perform optical measurements on each egg in the incubation tray and provide, for each egg, measured data indicative of radiation response of the egg to at least two illumination wavelengths;
a monitoring system configured and operable according to claim 1; and
a sorting utility configured to utilize the association data to control the sorting of the egg.

23. A sorting station for controlling sorting of fertile eggs progressing on a production line according to their genders, the sorting station comprising: a controller configured to be in data communication with the monitoring system of claim 1, and an identifier utility configured and operable to identify the egg and the association data of the identified egg, and generate sorting data defining further progress of the egg towards one of two routes.
